(19) United States
(12) Patent Application Publication
(10) Pub. No.: US 2012/0156744 A1
(43) Pub. Date: Jun. 21, 2012

(54) Multi-Phase, Gas-Lift Bioreactor for Generation of Biogas or Biofuel From Organic Material

(76) Inventors: Jeffrey M. Macdonald, Chapel Hill, NC (US); Andrey P. Tikunov, Chapel Hill, NC (US)

(21) Appl. No.: 13/388,686

(22) PCT Filed: Aug. 4, 2010
(86) PCT No.: PCT/US2010/044394

§ 371 Date: Mar. 9, 2012

Related U.S. Application Data
(60) Provisional application No. 61/231,133, filed on Aug. 4, 2009.

(51) Int. Cl. C12P 7/02; C12P 5/02; C12M 1/04

(52) U.S. Cl. 435/155; 435/294.1; 435/167

(57) ABSTRACT

Provided herein is a multi-phase, gas-lift bioreactor device for digestion and production of biogas or biofuel from organic material, as well as methods of use thereof.

### RELATED APPLICATIONS

This application claims the benefit under 35 U.S.C. §119(e) of U.S. Provisional Patent Application Ser. No. 61/231,133, filed Aug. 4, 2009, the disclosure of which is incorporated herein by reference in its entirety.

### FIELD

The present invention relates to a device for the digestion of organic material using microorganisms and methods of use of the same.

### BACKGROUND

Various designs of digesters exist for the processing and treatment of organic material, primarily organic wastes, to produce non-hazardous, and sometimes beneficial, products for use in low technology rural areas or for sophisticated industrial areas. Most digesters are based on either aerobic or anaerobic fermentation, and some combine elements of both.

One example of common organic waste is that produced by swine farms. Domesticated swine consume a controlled diet. Swine waste contains a large portion of small particles and primarily non-biodegradable cellulose from corn slurries in the diet, accounting for more than 50% of the potentially available methane (Boopathy, 1998).

Cellulose is a major component of swine feces representing as much as 30% depending on its original content in the diet (Kerr et al., 2006). The majority of thermophiles that have been discovered to digest cellulose are strictly anaerobic, making culturing challenging.

Successful anaerobic digestion of organic material usually requires a mixed culture of bacteria with a complex inter-dependency, terminating in the production of methane by methanogenic bacteria (Hawkes et al., 1987). Problems such as low methane yield and process instability are often encountered in anaerobic digestion, preventing this technique from being widely applied. A wide variety of inhibitory substances (ammonia, sulfide, light metal ions, heavy metals, and organics) are the primary cause of anaerobic digestor upset or failure since they are present in substantial concentrations in organic material such as wastes (Chen et al., 2008).

Many of the most common anaerobic methanogenic (i.e. methane generating) bacteria come from the kingdom Archea (Deppenmeier and Müller, 2006). Methanogens are widespread in anoxic environments such as fresh water sediments, swamps, tundras, rice fields, intestinal tracts of ruminants and termites, and anaerobic digesters of sewage treatment plants (Garcia et al. 2000). The products of methanogenesis, methane (CH₄) and carbon dioxide (CO₂), are greenhouse gases, and are of great interest for the global ecology (Deppenmeier and Müller, 2006). It is estimated that up to 20% reduction of global warming may be achieved by utilizing discarded biomass and waste for the production of biofuels and chemicals, outnumbering that of automobile and industrial contributions (Vieitez and Ghosh, 1999).

Presently, the majority of sewage systems, including swine waste sludge, undergo anaerobic digestion in holding ponds with these greenhouse gases escaping into the atmosphere. A recent publication from New Zealand which has over 1,000 wastewater stabilization ponds, found the biogas production from piggery and dairy ponds has a biogas (methane) production rate of 0.78 (0.53) m³/m²/d and 0.03 (0.023) m³/m²/d respectively, demonstrating that swine waste generates nearly an order-of-magnitude more methane than bovine waste (Park and Craggs, 2007). The difference is due to the higher indigestible cellulosic content of bovine waste versus swine.

Average CH₄ content of the piggery and dairy farm biogas were 72.0% and 80.3%, respectively. It was calculated that if the average volume of methane gas were captured from the piggery and dairy farm ponds (393.4 m³/d and 40.7 m³/d) and converted to electricity, this would reduce CO₂ equivalent green house gas emissions by 5.6 tonnes/d and 0.6 tonnes/d, respectively, and generate 1,180 kWh/d and 122 kWh/d, respectively, further demonstrating the order-of-magnitude higher methane generation that from swine versus bovine. An Australian study found that well-managed piggery farm with 15,000 pigs could save 6,852 to 8,015 tons of CO₂ equivalence per year, which equates to the carbon sequestrated from 6,800 to 8,000 spotted gum trees (age=35 year) in their above plus below-ground biomass (Maraseni and Maroulis, 2008). In short, there is a considerable amount of green house gas with great potential for energy recovery (Park and Craggs, 2007).

More methods are needed to efficiently digest and dispose of organic material and preferably provide beneficial end products such as harvestable methane and biofuels.

### SUMMARY

Provided herein is a multi-stage plug-flow gas-lift digestion device for the digestion of organic material.

In some embodiments, the device includes: a stage one digestion module, including i) a first digestion vessel having an inlet, an outlet, and a first digestion chamber therebetween, said digestion chamber having an upper portion and a lower portion; ii) at least one flow tube positioned in said first digestion chamber and dividing said chamber into an inner lumen and an outer lumen, said flow tube configured to allow current flow between said outer lumen and said inner lumen; and iii) a first gas source connected to said digestion chamber and configured to bubble a first gas in each of said at least one flow tubes to create a gas-lift flow in said inner lumen.

In some embodiments, the device includes: a stage two digestion module, including i) second digestion vessel having an inlet, an outlet, and a second digestion chamber therebetween, with said second digestion vessel inlet in fluid communication with said first digestion vessel outlet, said second digestion chamber having an upper portion and a lower portion; ii) at least one flow tube positioned in said second digestion chamber and dividing said chamber into an inner lumen and an outer lumen, said flow tube configured to allow current flow of said organic material between said outer lumen and said inner lumen; and iii) a second gas source connected to said second digestion chamber and configured to bubble a second gas in each of said at least one flow tubes to create a gas-lift flow in said inner lumen.

In some embodiments, the at least one flow tube is configured to allow current flow of said organic material from the outer lumen to the inner lumen at the first digestion chamber bottom portion, and current flow from the inner lumen to the outer lumen at said first digestion chamber upper portion. In some embodiments, the current flow is laminar flow.

In some embodiments, the first gas source is connected to the first digestion chamber lower portion beneath each of said at least one flow tubes to create a gas-lift flow in said inner lumen. In some embodiments, the second gas source is connected to the second digestion chamber lower portion beneath each of the at least one flow tubes to create a gas-lift flow in said inner lumen.

In some embodiments, the stage one digestion module and/or stage two digestion module further includes a foam collector configured to accept foam that develops on the surface of the organic material at the top portion of the at least one chamber. In some embodiments, the foam collector includes a foam outlet and a gas inlet, and the stage one digestion module and/or stage two digestion module further includes a foam separator in fluid communication with the foam outlet, the foam separator including a top portion and a bottom portion, the top portion having a gas outlet in gas communication with the gas inlet of the foam collector, and the bottom portion having a liquid outlet, the liquid outlet in fluid communication with the first or second digestion vessel.

In some embodiments, the device includes a water jacket surrounding the first digestion vessel and/or the second digestion vessel. In some embodiments, the device further includes a heater operatively connected to a water jacket.

In some embodiments, the device further includes a heat-exchanger operatively associated with the first gas source and configured for distillation of said first gas, and/or a heat-exchanger operatively associated with the second gas source and configured for distillation of the second gas.

In some embodiments, the stage one digestion module and/or stage two digestion module includes at least three digestion vessels arranged in series.

A method of digesting organic material is also provided, the improvement comprising digesting said material using the device of any of the preceding paragraphs.

A method of digesting organic material and creating methane is provided, including the steps of: 1) providing a multi-stage plug-flow gas-lift digestion device of any of the preceding paragraphs; 2) loading the organic material into the inlet of the stage one digestion module; 3) mixing the organic material by bubbling the first gas in the first reaction vessel; 4) overfilling the reaction chamber of the first reaction vessel so that the organic material spills into one or more subsequent chambers, the bacterial mixture digesting the organic material to create acetate, and the acetate created thereby flowing into the first module outlet; 5) passing the acetate and the bacterial mixture through the stage one digestion module outlet and into the stage two digestion module inlet; 6) mixing the acetate by bubbling the second gas in second reaction vessel; and 7) overfilling the reaction chamber of the second reaction vessel so that acetate spills into a subsequent chamber or outlet, the bacterial mixture digesting the acetate to form the methane.

In some embodiments, the methods further include providing in the device and/or in the organic material a bacterial mixture capable of digesting the organic material and capable of producing methane from acetate.

In some embodiments, the method further includes providing conditions (e.g., temperature, pH) in the stage one digestion module conducive to digestion of the organic material by the bacterial mixture; and providing conditions (e.g., temperature, pH) in the stage two digestion module conducive to production of the methane by the bacterial mixture from the acetate.

In some embodiments, the method further includes: (a) alkalinizing the organic material to thereby saponify ester linkages therein; (b) acidifying the organic material to thereby hydrolyze peptide linkages; (c) heating the organic material to facilitate the digesting; (d) exposing the organic material to electromagnetic energy to break aromatic linkages in the organic material; or (e) combinations thereof.

Also provided is a method of collecting a biofuel (e.g., an alcohol such as ethanol or butanol), including the steps of: 1) providing a multi-stage plug-flow gas-lift digestion device which has a heat-exchanger; 2) loading the organic material into the inlet of the stage one digestion module; 3) mixing the organic material by bubbling the first gas in the first reaction vessel; 4) overfilling the reaction chamber of the first reaction vessel so that the organic material spills into one or more subsequent chambers, the bacterial mixture digesting the organic material to create acetate, and the acetate created thereby flowing into the first module outlet; 5) passing the acetate and the bacterial mixture through the stage one digestion module outlet and into the stage two digestion module inlet; 6) mixing the acetate by bubbling the second gas in second reaction vessel; and 7) overfilling the reaction chamber of the second reaction vessel so that acetate spills into a subsequent chamber or outlet, the bacterial mixture digesting the acetate to form the methane, and 8) collecting the distillate from the heat-exchanger, which distillate includes the biofuel.

In some embodiments of the methods of any of the preceding paragraphs, the organic material may include municipal, industrial, agricultural or domestic wastes. For example, the organic material may include bovine or swine fecal wastes.

It will be understood that all of the foregoing embodiments can be combined in any way and/or combination.

### BRIEF DESCRIPTION OF THE DRAWINGS

FIG. 1 is a diagram illustrating some common stages of anaerobic conversion.

FIG. 2 is a block diagram illustrating a two-phase gas-lift bioreactor according to some embodiments. Each chamber in the module has a tube (3), which divides it in two connected coaxial volumes: the central volume or tube, has a gas jet in the bottom of each chamber (2) through which gas bubbles pass creating an upward suction in the central volume, the downward convection in the peripheral volume and together the entire volume is filled with carrier, providing a surface for bacterial growth and continuous convection currents circulating between the central and peripheral volumes.

FIG. 3 is a block diagram illustrating an overall bioreactor functional scheme according to some embodiments. This embodiment exemplifies the gas line entry going down into the chambers from the top of the module.

FIG. 4 is a schematic diagram illustrating the bioconversion of swoop in the two modules according to some embodiments.

FIG. 5A is a digital image of an embodiment of the bioreactor.

FIG. 5B is a graph of the results of E. coli in glucose solution and the comparison of growth with a stirred bioreactor.

FIG. 6 is a schematic diagram illustrating the general structure of an embodiment of the two-phase gas-lift bioreactor. This depicts just one of the two modules (dark, and second module or phase (light) and a duplicate of the first system minus the grinder).

FIGS. 7A-B are graphs of the heat capacity of various materials control the time for cooling from 70° C. to 55° C. From top to bottom: blue line: 70 kg of water; black line: cement; red line: ceramic; gray line: aluminum.

FIG. 8 is a schematic diagram of an embodiment of the gas-lift modular bioreactor that includes a pretreatment module connected to the two-phase bioreactor. Examples of mechanical, chemical and physical digestions are included in the pretreatment module, and may be incorporated as desired. As shown, in some embodiments there may be a connection between the pretreatment module and the second module to allow smaller material to bypass the phase one digestion, while the larger material will fall to the connection with the phase one treatment module.

FIG. 9A is a digital image of a single module of a gas-lift bioreactor according to some embodiments.

FIG. 9B is a graph of the results of swine waste bacteria in 1% w/w boiled corn suspension and the comparison of oxygen consumption with a constantly stirred bioreactor.

FIG. 10 is a graph of the ¹H NMR spectrum of condensate, obtained from gas-lift bioreactor, anaerobically processed 1% w/w boiled corn (7th day). Butanol co-resonates with ethanol (3.6 ppm) and lipids (0.89 ppm) are the small peaks at 1.34 and 1.51 ppm.

### DETAILED DESCRIPTION OF THE PREFERRED EMBODIMENTS

Provided herein is a multi-phase, gas-lift bioreactor device for digestion and production of biogas from organic material. The device is multi-chambered and in some embodiments has a sequential series of reaction chambers. The gas-lift feature provides flow within the reaction chambers and through the device without the need for internal moving parts. The disclosures of all patent references cited herein are to be incorporated by reference in their entireties.

“Multi-phase” or “multi-stage” as used herein refers to the separation of at least two main stages (or “phases”) of digestion of organic material: 1) breakdown of macromolecules by hydrolysis via biocatalyst or chemical acidification or alkalization; and 2) further chemical modification of the molecules via acetification and methanogenesis, into at least two distinct regions of the digestion device. Each of these stages of digestion require different optimal ranges of conditions (e.g., temperature, pH, etc.), and therefore their separation allows better operation of each stage. See U.S. Pat. No. 5,525,228 to Dague et al.; U.S. Pat. No. 5,637,219 to Robinson et al.; U.S. Pat. No. 6,673,243 to Srinivasan et al. Inclusion of these two main stages is sometimes referred to as “two-phase” or “two-stage.” However, the use of the term “two-phase” or “two-stage” herein does not preclude the use of additional stages and/or modules of digestion in embodiments of the present invention.

In a “plug-flow” digester, the organic material passes through the digester in a sequential manner from the inlet to the outlet. Typically, the design is tubular and unstirred. The solid material tends to move through the plug-flow digester sequentially, while the liquid fraction mixes more rapidly. See U.S. Pat. No. 6,673,243 to Srinivasan et al.

“Gas-lift” is the use of a flow of gas to effect movement, or flow, of liquid by, e.g., bubbling. In some embodiments, the gas is an anaerobic gas such as nitrogen or a mixture comprising nitrogen. In some embodiments, the gas lift effects a laminar flow of the liquid in digestion chambers of the bioreactor, the fluid flowing smoothly in parallel layers with a parabolic velocity profile. However, in some embodiments, the gas lift may effect a turbulent flow, by, e.g., increasing the flow velocity, including an obstacle, if it is desired to break apart the material in the digestion chamber.

Any energy source may be used to power the device. In some embodiments, energy may be provided with a Tesla turbine, which is known to be highly efficient and durable, and can be used with gas, foam or liquid. The Tesla turbine in some embodiments is powered by steam produced by burning the biogas (e.g., methane, hydrogen, etc.) or other biofuel produced by the digestion process, and can generate electricity that may be harvested.

“Organic material” as used herein refers to complex organic molecules (i.e., molecules including atoms of carbon and hydrogen), often derived from living, or recently living organisms. Many types of organic material and/or waste, e.g., municipal, industrial, agricultural and domestic wastes, may be digested with the device and methods as taught herein. Examples include, but are not limited to, organic waste (e.g., animal wastes such as feces, carcasses or wastes from animal husbandry or meat processing), cellulosic material such as wood or paper, beer, corn, crops, or produced agricultural biomass such as potato, miscanthus, switchgrass, hemp, corn, poplar, willow, sorghum, sugarcane, eucalyptus, palm oil, etc.

Examples of animal wastes that may be digested by the methods taught herein include sludge from wastewater sumps from animal or agricultural operations such as bovine, swine, or digester sludge from municipal wastewater treatment systems. As used herein, “swoop” is organic fecal waste from swine, which is an example of organic material that may be processed as taught herein.

The organic material may be mixed with a carrier such as water to allow more efficient flow in the bioreactor as taught herein, and may include liquid material, solid material, or a mixture of both, and the digestion parameters may be optimized based upon the molecule content of the organic material to be processed as taught herein and as generally known to those of skill in the art.

“Digestion” as used herein refers to the conversion of more complex organic molecules into smaller chemical entities. This may be accomplished, for example, through the use of living “microbes,” typically bacteria (“bacterial digestion”); through the use of chemicals (e.g., a basic solution (saponification), enzymes etc.) (“chemical digestion”); the use of physical energy (e.g., heat or electromagnetic energy), etc., or combinations thereof. For example, fats, proteins and carbohydrates such as cellulose may be digested by bacteria to form fatty acids, amino acids and sugars, respectively. These small chemical entities may, in turn, be digested into even smaller entities, such as acetate, formate, propionate, butyrate, hydrogen and carbon dioxide.

However, it should be understood that molecules could also be built up to larger molecular weight and then degraded, or not, as desired by use of the bioreactors described herein.

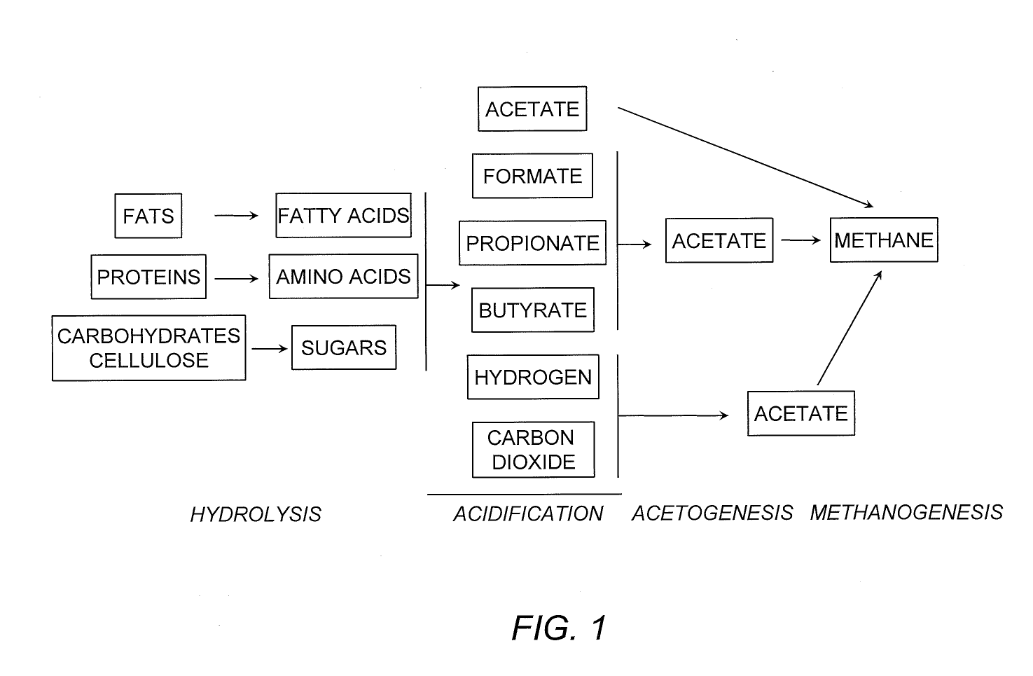

The scheme presented in FIG. 1 illustrates the common stages of anaerobic conversion that may be used to digest organic material according to some embodiments.

The microbiology of anaerobic digestion can be generally described as including four broad trophic groups, which digests organic materials in sequence. The first group, the hydrolytic and fermentative bacteria, contains obligate and facultative anaerobes, and removes small amounts of oxygen that may be introduced into the digester with the organic material influent. By hydrolysis, this group initially breaks down the more complex molecules (e.g., cellulose, starch, proteins, lipids, etc.) into smaller units (e.g., amino acids, sugars and fatty acids). Then, by a process of acidification, this group uses these smaller compounds to produce formate, acetate, propionate, butyrate, hydrogen and carbon dioxide. These acidic products are then available for the next trophic level.

In many digesters, the rate-limiting step in the hydrolysis of complex molecules, particularly the polysaccharides, is the disaggregation and then biocatalytic digestion to micromolecules (Hawkes et al., 1987). Therefore, in some embodiments, a chemical and/or physical digestion is included (e.g., prior to bacterial digestion), such as alkalinization of organic material to saponify the ester linkages of the fats, decreasing the complexity of the macromolecular component and degrading lipids to free fatty acids or proteins to smaller peptides, leaving primarily polysaccharides as the primary insoluble fraction. This could include high heat and acid conditions to digest peptide linkages in proteins. In some embodiments, electromagnetic energy may be used, e.g., in the ultraviolet range, which would break aromatic linkages. This may be especially useful in decreasing the xenobiotic load (pesticides, drugs, etc.) of the material, degrading aromatic compounds to smaller alkyl compounds.

In some embodiments, the organic material may be physically broken apart and/or homogenized prior to digestion. For example, the organic material may be homogenized in a grinder prior to digestion according to some embodiments. In some embodiments, a sonicator may be used to homogenize the organic material. The gas-lift flow may also be used to create turbulent flow to break apart the material according to some embodiments.

The second trophic group comprises hydrogen-producing acetogenic bacteria, or proton-reducing bacteria. By a process of acetification (also called acidification), this group makes acetate from compounds such as fatty acids, buturate, formate and propionate. The third trophic group of bacteria (which may or may not be used) comprising homoacetogenic bacteria, produces acetate from hydrogen gas and carbon dioxide. The final trophic group comprises the methanogenic bacteria, which convert compounds such as acetate into methane gas and carbon dioxide in a process called methanogenesis. This group is strictly anaerobic, requiring an oxygen-free environment.

Thermophiles (grow optimally from 55-70° C.) and extreme thermophiles (grow at >70° C.) both show extensive promise for use in biogas production, and are capable of degradation of a range of substrates (Blumer-Shuette et al., 2008; Nercessian et al., 2005). Complex degradation processes of organic matter are performed by fermentative and syntrophic bacteria (Schink, 1997), ending in small molecules such as formate, methylamines, and methylated thiols, which serve as substrates for methanogenic bacteria. It has been suggested that the production of methane from cellulose cannot be highly efficient from a single bacterium, but rather requires multiple species that include the ability to biochemically process intermediates, including acidogenic and acetogenic bacteria that can breakdown cellulose metabolites into energy sources suitable for methanogenesis. Some microbes will digest the cellulose, while others are better at degrading the lipids and proteins. Also, the biotope will be digested to soluble monomer, such as glucose, while others may degrade to gas, such as methane (Deppenmeier and Müller, 2006) or hydrogen (Chou et al., 2008).

In some embodiments, mixtures of microbes are added to the organic material prior to digestion. In some embodiments, microbes are provided in the device, e.g., seeded in the device prior to digestion (see U.S. Pat. No. 5,637,219 to Robinson et al.) immobilized on matrices (see U.S. Pat. No. 6,254,775 to McElvaney). In some embodiments, the organic material contains adequate microbes which are native to the organic material, and additional microbes need not be added (see U.S. Pat. No. 6,673,243 to Srinivasan et al.). In some embodiments, Fe (III) and/or Geobacter strain NU may be added to degrade odoriferous volatile fatty acids and increased methane production (Coates et al., (2005), Biological control of hog wastes odor through stimulated microbial Fe(III) reduction, Appl. Environ. Microbiol. 71: 4728-4735).

In some embodiments, “biofuel” may be harvested from the device, e.g., a “biogas” such as methane, hydrogen, etc., or other biofuels such as ethanol or butanol.

Swine waste, for example, has over 100 endogenous forms of volatile biofuel from parafins, olefins, aromatics, ethers, alcohols, aldehydes, ketones, phenols, halogenate hydrocarbons, and sulfides (Blunden et al., (2005), Characterization of non-methane volatile organic compounds at swine facilities in eastern North Carolina, Atmospheric Environment 39(36): 6707-6718.). Biofuels have been generated from swine waste by high energy treatment with microwaves (Li et al., (2009), Comparison of saccharification process by acid and microwave-assisted acid pretreated swine manure, Bioprocess and Biosystems Engineering 32(5): 649-654), hydrothermal induction free fatty acid saponification (Xiu et al., (2010), Effectiveness and mechanisms of crude glycerol on the biofuel production from swine manure through hydrothermal pyrolysis, Journal of Analytical and Applied Pyrolysis 87(2): 194-198), addition of glucose and anaerobic digestion (Wu et al., (2009), Continuous biohydrogen production from liquid swine manure supplemented with glucose using an anaerobic sequencing batch reactor, International Journal of Hydrogen Energy 34(16): 6636-6645). Ethanol production as even been accomplished by growing duckweed on swine waste (Cheng et al., (2009), Growing Duckweed to Recover Nutrients from Wastewaters and for Production of Fuel Ethanol and Animal Feed, Clean-Soil Air Water 37(1): 17-26). However, all these require far more effort and energy than the use of the bioreactors described herein, whereby, in some embodiments, the bioreactor can be heated with the co-generation of heat from the biogas generator, and distilled simultaneous and in parallel with methane production.

In some embodiments, ethanol production may be further increased by the addition of yeast and/or cellulose degrading thermophiles. For example, a highly active cellulose producing microbe such as that recently isolated from swine waste may be used in the digestion according to some embodiments to make glucose and then ethanol (Liang et al., (2010), Toward Plant Cell Wall Degradation Under Thermophilic Condition: A Unique Microbial Community Developed Originally from Swine Waste, Applied Biochemistry and Biotechnology 161(1-8): 147-156). Thermophilic organisms are generally regarded as preferable with respect to hydrogen production rates and yields for cellulose degradation (Claassen et al., (1999), Utilisation of biomass for the supply of energy carriers, Appl. Microbiol. Biotechnol. 52: 741-755).

There are several sets of anaerobic thermophiles that are active in cellulose degradation. For example, organisms such as Clostridium thermocellum, C. cellulosi, Thermoanaerobacter cellulolyticus, and Anaerocellum thermophilum have all been reported to have the capacity for cellulose degradation (Rainey et al., (1993), 16S rDNAa nalysis reveals phylogenetic diversity among the polysaccharolytic Clostridia, FEMS Microbiol. Lett. 113: 125-128), as well as Thermatoga elfii, Caldicellulosiruptor saccharolyticus, and Clostridium sp. strain JC3 (Syutsubo et al., (2005), Behavior of cellulose-degrading bacteria in thermophillic anaerobic digestion process, Water Sci. Tech. 52: 79-84). Substrate concentration, pH, and other coenzymes and cofactors may be adjusted as needed to optimize metabolic efficiency.

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

### EXAMPLES

### Example 1

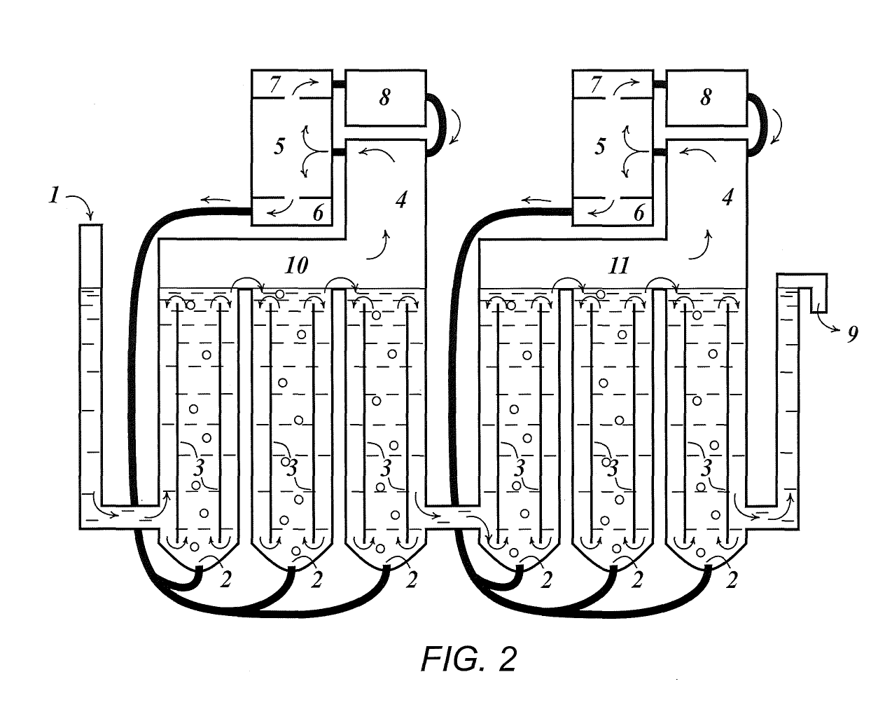

To start, the bioreactor is filled with water and mixing is created by the gas raising up through the center tube (FIG. 2, 3) creating a convection current around the central volume (FIG. 2, 3) (see U.S. Pat. No. 642,460). Liquid organic material such as swine waste (i.e., swoop) loads through the grinder into the loading column, goes to the first chamber, mixes there with anaerobic carrier gas (e.g., N₂) and when the chamber overfills, the solution or sludge falls into second chamber, and so on. The third chamber of the first module is connected with first chamber of the second module by tube in the bottom part connecting the two modules, so material reaches the second module with extensive mechanical disruption of the material, while permitting isolation of the gas mixtures and temperature within of these modules. Carrier gas (e.g., N₂ propane (see U.S. Pat. No. 5,651,890) or any desired gas mixture) provides mixing of suspension and at the same time provides necessary supplements and activates organic material digestion. Gas passes through the organic material suspension and forms the foam, consisting primarily of fats and denatured proteins. This foam digests in the foam-chamber (FIG. 2, 4) and partially goes to the foam separator (FIG. 2, 5) where half of gas with foam goes back to bioreactor via lower Tesla turbine (see U.S. Pat. No. 1,061,206 to Nikola Tesla) (FIG. 2, 6) and the other half goes through an upper Tesla turbine (FIG. 2, 7) to the gas manipulator (FIG. 2, 8), which consists of all or a combination of a sparger, a gas analyzer, a collector, condenser for distillation of volatile compounds, and an exchanger. In the manipulator, supplemental gases are mixed with external gases to create an optimum gas mixture for digestion, or the gas from the bioreactor could be taken and substituted with carrier gas such that the same volume enters the foam-chamber (FIG. 2, 4) and exits the foam separator (FIG. 2, 5) by way of the gas-jet bottom (FIG. 2, 2). Each module may be water-jacketed and may have an isolated gas-recirculating system, which can be maintained at a desired environmental condition (temperature, aerobic/anaerobic gases) in the two connected modules as if they were isolated.

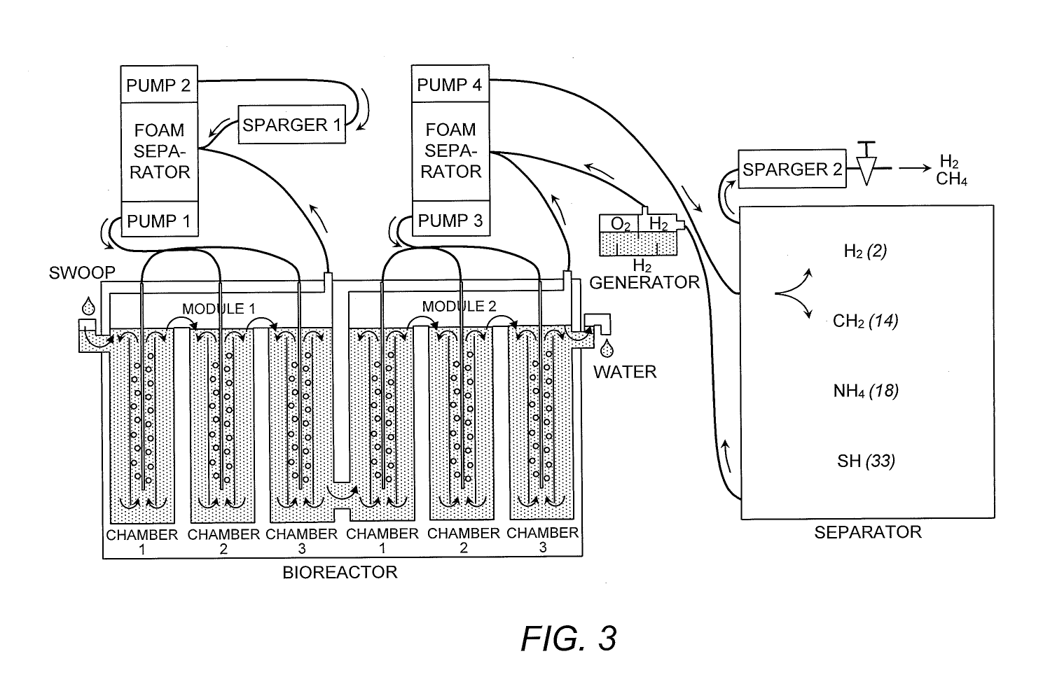

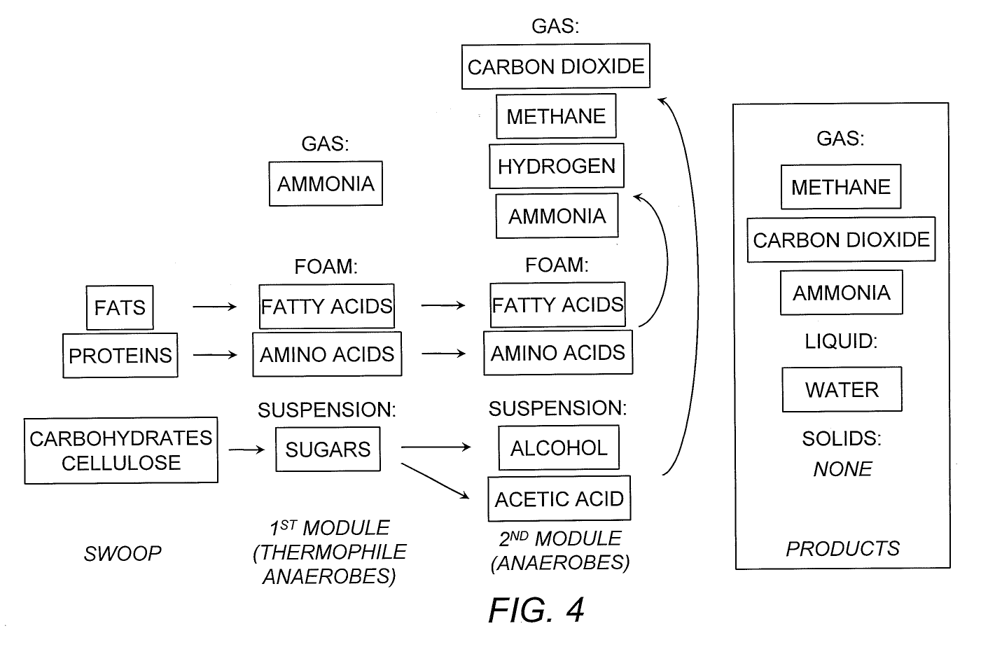

Overall, the two phases of the bioreactor are referred to herein as module 1 and 2 (FIGS. 2, 10 and 11, respectively). FIG. 3 is a schematic diagram of the two modules and the various degradation products of organic material generated in the respective modules. Module 1 is designed to separate the organic material (FIG. 3, 1) into its lipophilic fraction, which forms foam on the top of the water, and cellulose particulate matter that circulates and degrades. In Module 2, the dissolved small molecules that were broken down from the macromolecules in module 1 are metabolized to methane and hydrogen gas. FIG. 3 shows the phase separation, which involves two aspects: two modules of reactor (called module 1 and 2 in FIG. 3 which corresponds to phase 1 and 2) physically separate processes of macromolecules degradation (module 1, FIG. 3) and methane generation (module 2, FIG. 3). The second aspect of phase separation is mechanical isolation and concentration of the lipophilic fraction at the top of module 1 and to a much lesser extent in module 2 (FIG. 3). Being hydrophobic, lipids extensively foam when bubbled with gas, and accumulate in the foam separator chamber. This separation of substrates, processes and products allow adjustment of the conditions for optimization of each step of organic material degradation and biogas production.

This bioreactor design permits adjustment of the following environmental parameters:
1. Temperature of cellulose degradation and biogas or biofuel production in phase 1 and 2 digestion, respectively. It is well established that temperature has effects on growth rate kinetics (Zinder et al., 1984). Efficiency of the system is expected to increase with the increase in temperature until it reaches a peak at approximately 75° C. for phase 1 and approximately 53-60° C. for the second phase.
2. Loading rate. Because of plug-flow operation, in some embodiments constant inflow of the substrate (organic material) may be needed to drive the degradation processes. This flow-rate will determine digestion time in phase 1 and 2. A typical loading rate for mesophilic reaction is one bioreactor volume per 20-30 days, for thermophilic reaction digestion time is one bioreactor volume per 10-15 days. Preliminary data show that only gas-lift mixing and extreme thermophiles should increase bioreactor efficiency 2-4 times. Therefore, loading rate according to some embodiments is expected to be one bioreactor volume per 2-5 days.
3. First to second phase volume ratio. Cellulose degradation rate is expected to be slower than methane production, so digestion time in phase 1 may be increased in some embodiments by addition of the extra chambers to the first phase of the bioreactor reactor taking advantage of the modularity of the design.
4. Intensity of gas-lift mixing. Gas-lift is a highly efficient mixing method. Optimal gas bubbling speed can also be determined to provide necessary and economical mixing. However, excessive gas-lift intensity might subject bacteria to shear forces that could inhibit growth.

To summarize, some embodiments of the multi-phase gas-lift bioreactor is advantageous because the first phase of the digestion can be optimized for temperature and as content for the microbes involved in macromolecule degradation, while the second phase can be optimized for biogas or biofuel generation.

In general, organic material extensively foams when bubbled with gas, so in some embodiments the first phase of the bioreactor has the added function of mechanically separating the material, such that two populations and environments can be cultured: one in suspended in the solution metabolizing the cellulose, the second on the surface in hydrophobic environment degrading lipids.

Although swoop and boiled corn were used in demonstrating the system, any organic material containing solid and liquid fractions could be used for digestion.

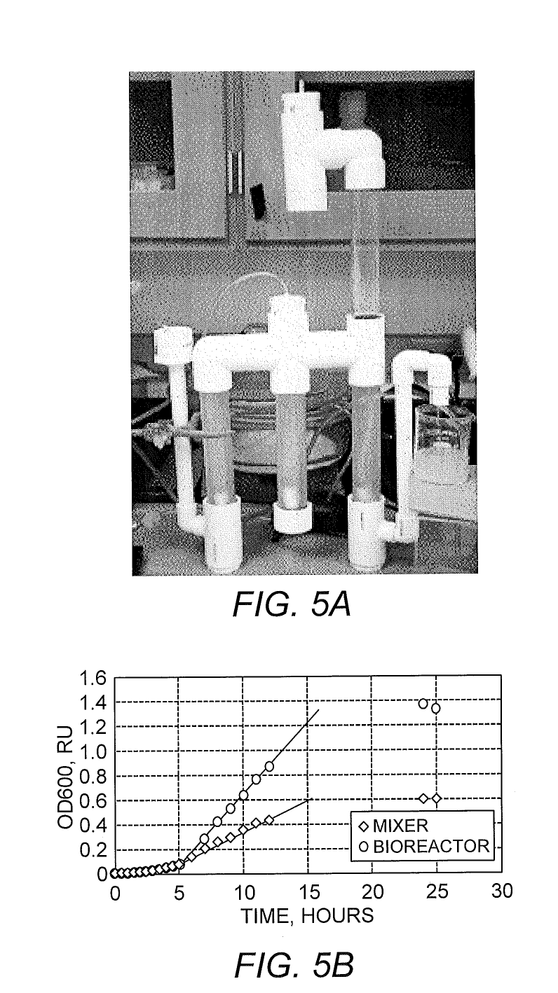

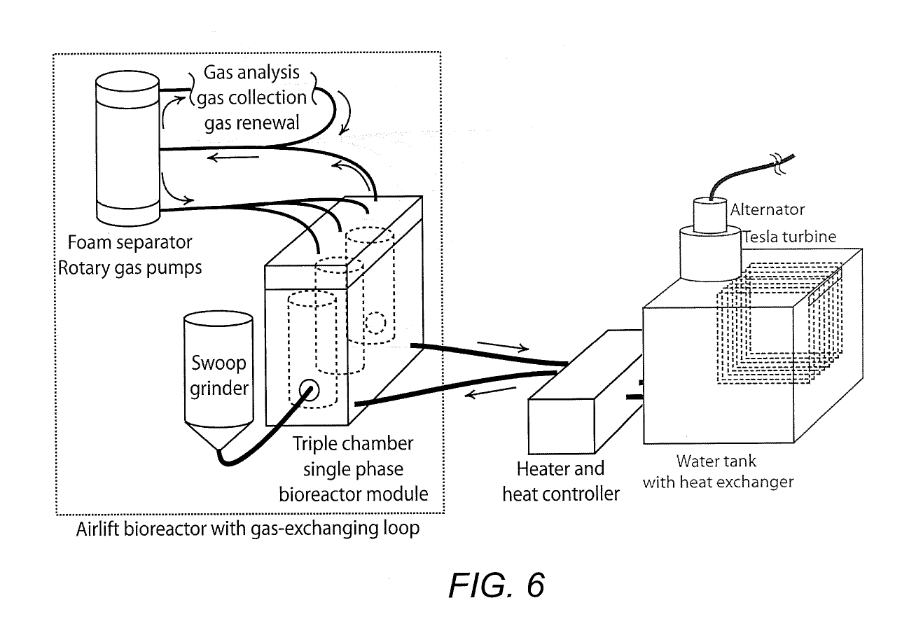

Data from the initial experiments with the plug-flow, gas-lift bioreactor confirm that the performance is 3-fold greater than a stirred bioreactor using E. coli and glucose as a substrate. FIG. 5A is a digital image of an embodiment of just one of the modules (FIG. 3) of the bioreactor and uses relatively inexpensive material that is typically readily available. In the results of the study shown in FIG. 5B, the bioreactor was acting as it would in phase 2, whereby small molecular weight breakdown products from phase 1 macromolecular degradation, are metabolized to end products. In this case, the system is working in an aerobic mode and lactate is the breakdown product. The growth of the bacteria was measured in a stirred beaker representing the stirred bioreactor, and growth was quantified by the percent of transmission of light through the solution, which normally would be clear in pure glucose solution, but become cloudy with bacterial growth. The least-squares best fit of the time course shows the plug flow, gas-lift bioreactor outperformed the stirred bioreactor by 2.5-fold.

### Example 2

In some embodiments, it is preferable to maintain high temperature and reduce temperature oscillations. Constant temperature may be supplied via selection of materials with appropriate heat capacitance (thermal mass). Heat capacitances of various materials are shown in Table 1:

TABLE 1

| Material | Heat capacity, kJ/m³° C. |
|---|---|
| Aluminum | 0.87 |
| Firebrick | 1.05 |
| Cement dry | 1.55 |
| Water | 4.2 |

The temperature dependence over time is given by:

$$T ( t ) = Ta + ( Ti - Ta ) {e}^{- \frac{α S}{cm} t}$$

Where: c—heat capacity (thermal mass), m—mass (approx 70 kg), α—coefficient of heat transfer (for 10 cm of phenolic foam insulation it is 0.2 W/m ° C.), S—square of heat exchange (approx 1 in²), Ti—initial temperature, Ta—ambient temperature (average annual ambient temp for Raleigh, N.C. is 13.3° C.).

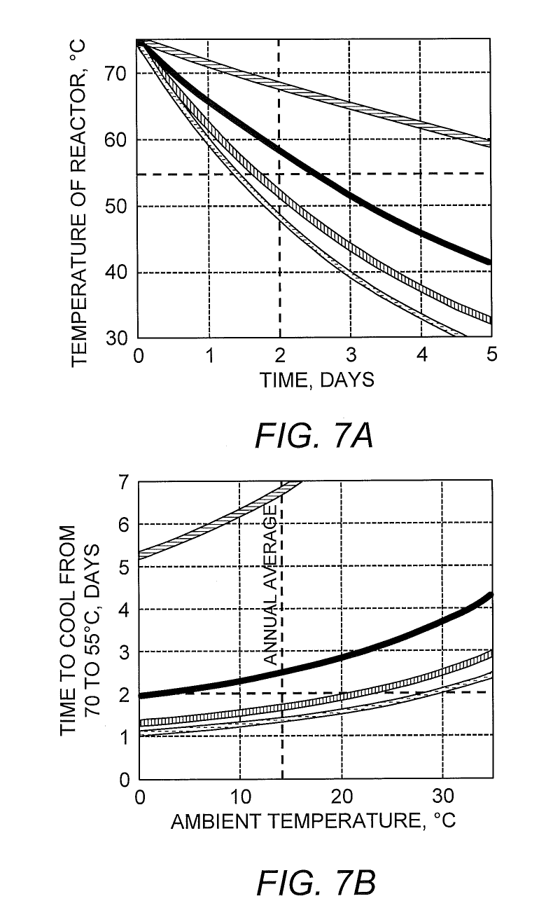

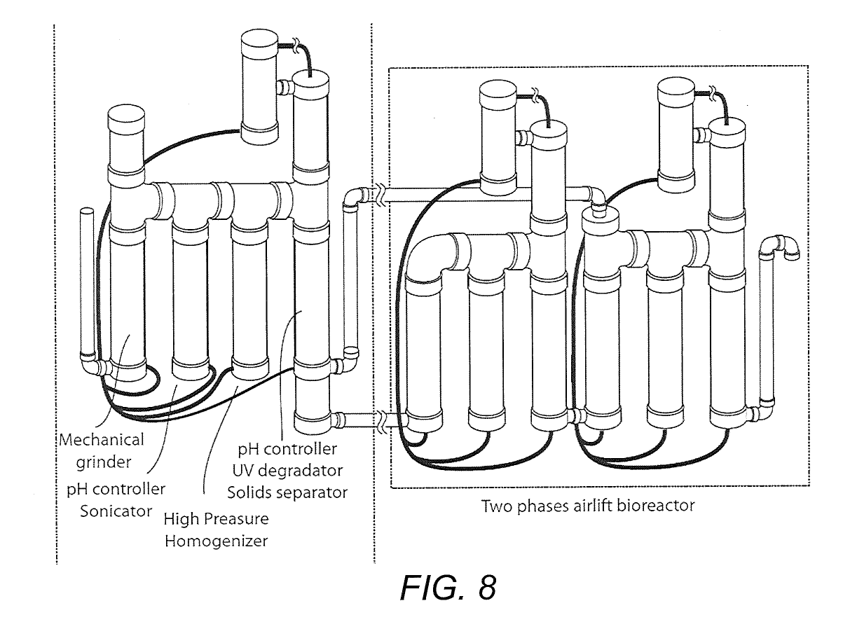

Curves of temperature changing over time are shown in FIG. 7A for various materials. The lowest temperature for thermophiles to grow is about 55° C., which is shown with a dashed horizontal line.

The average annual sunny days in Raleigh, N.C. are 111 days per year (this does not include partly sunny or partly cloudy days). On average each third day is sunny, so the material comprising the bioreactor to maintain its temperature above 55° C. for two days without any external heating (shown with vertical dashed line). Cement may maintain the temperature for about 2.5 days, ceramic and aluminum—for approximately 1.5 days, and 70 kg of water in the same conditions may maintain temperature for more than 5 days. These calculations are made for an average annual ambience temperature in Raleigh, N.C. area (13.3° C.), but temperature may vary from −1 to 31° C.). It is known that the speed of cooling is the reciprocal of the ambient temperature. FIG. 7B shows the time needed to cool the bioreactor from 70 to 55° C. depending on ambient temperature. As illustrated in FIG. 7B, the bioreactor made of cement can keep necessary temperature during 2 days and longer in the whole annual temperature range. The calculations show that the bioreactor module with mass approximately 70 kg, made of cement or with 70 kg water heat buffer and with 10 cm of thermoisolation can maintain temperature needed for thermophiles for 2 days or longer without any external heating.

In some embodiments, it is more efficient and inexpensive to use water as the heat exchanging material than cement. In this case, different modules can share a heater and tank with hot water used as heat capacitor. The heat system of the bioreactor operates as it shown in FIG. 5. Water is heated in a water heat buffer (potentially, water temperature may be up to 95° C.). The heat controller has a water pump and a set of valves, and water is circulated around the bioreactor via water jackets. A thermocouple may measure temperature of the bioreactor core and will mix the water in the jacket with hot water from the heat buffer upon cooling. The heat controller may have a small electrical heater to maintain minimal necessary temperature in bioreactor only in emergency cases, or if water heat buffer cools down to the crucial temperature it isolates from bioreactor.

### Example 3

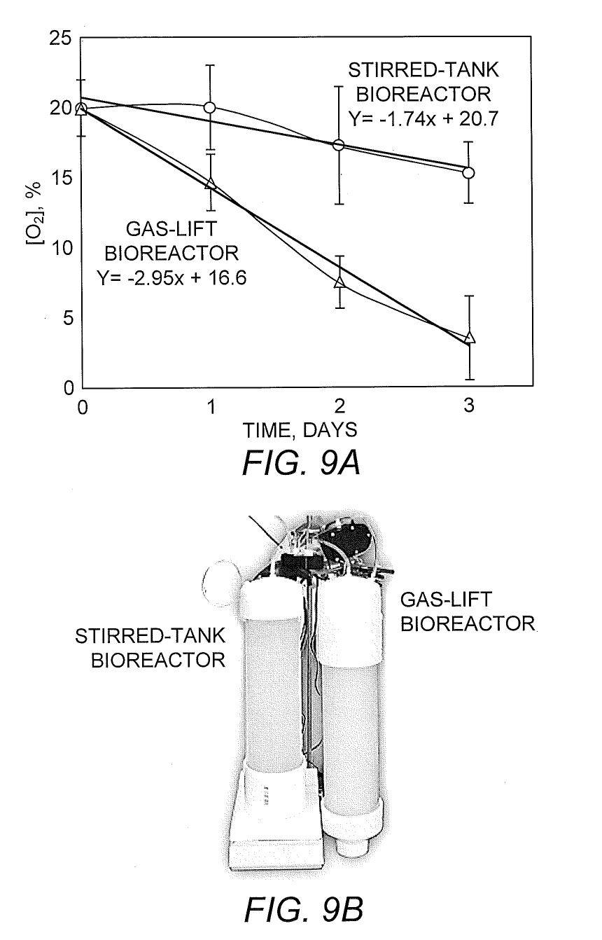

Experiments with a single gas-lift bioreactor module demonstrated that the performance is approximately 2-fold greater than a stirred bioreactor using swine manure as a bacterial source and 1% w/w boiled corn as a substrate. Two bioreactors side-by-side are shown in FIG. 9A. On the left is a constant-stirred tank bioreactor driven by magnetic stirrer. On the right is the embodiment of just one of the chambers (see FIG. 5) of the bioreactor using relatively inexpensive material.

In the results of the study presented in FIG. 9B, the bioreactor performed as typical in phase 1, where bacteria naturally presented in swine waste degrade macromolecules (sugars, lipids, protein) to small molecular weight breakdown products (mostly acetate).

Initially, both bioreactors were started under aerobic conditions, simulating loading of fresh organic waste to the first phase. Gas volumes in the bioreactors were isolated from the external atmosphere so that oxygen consumption by presented bacteria led to significant drop in [O₂] in the bioreactor gas volume. The oxygen concentration was measured by Clark-type platinum-silver electrodes. The least-squares best fit of the time course shows the gas-lift bioreactor utilizes oxygen ˜2-fold faster than the stirred bioreactor, which indicates faster bacterial growth due to better gas exchange.

One of the potential advantages of some embodiments of the gas-lift bioreactor described herein (besides better mixing and lack of moving parts) is constant gas flow through the digestion mixture, which saturates gas with vapors (water vapor and any volatile component contained in mixture, e.g. ethanol). Addition of a heat-exchanger to the gas-pumping system allows the distillation of pure water containing condensated volatiles. In turn, this strips the gas of vapor, significantly increasing ethanol/water solubility in the subsequent pass through the bioreactor.

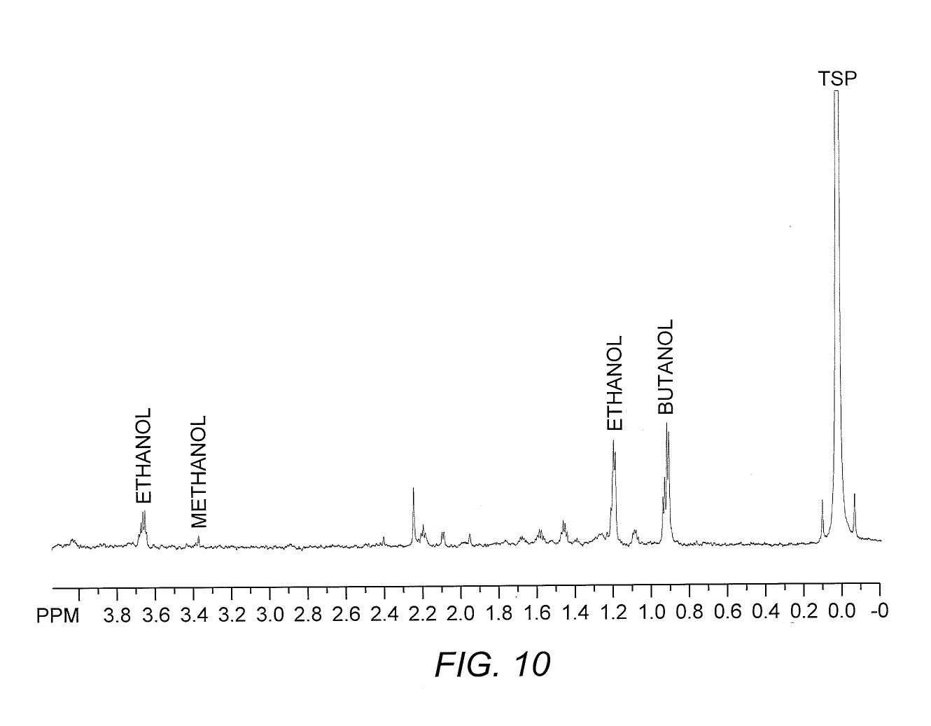

FIG. 10 is a ¹H NMR spectrum of the condensate of a bioreactor run outline in FIG. 9. This experiment was performed under anaerobic conditions used 1% (dry weight/weight) of boiled corn as a substrate for mixture of native swine waste bacteria (no artificial yeast was added). In this preliminary experimental setup without any optimization of conditions and microbial population, the condensate obtained from the gas-lift bioreactor running under anaerobic conditions, contains millimolar concentrations of ethanol and lower concentrations of butanol (note figure legend for butanol peak assignment). Note that the condensate can be obtained without any additional operational costs just by addition of a simple heat-exchanger and maintaining 10° C. lower temperature. No boiling of fermenting mixture was necessary.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

1. A multi-stage plug-flow gas-lift digestion device for the digestion of organic material, comprising:
(a) a stage one digestion module, comprising:
i) a first digestion vessel having an inlet, an outlet, and a first digestion chamber therebetween, said digestion chamber having an upper portion and a lower portion;
ii) at least one flow tube positioned in said first digestion chamber and dividing said chamber into an inner lumen and an outer lumen, said flow tube configured to allow current flow between said outer lumen and said inner lumen; and
iii) a first gas source connected to said digestion chamber and configured to bubble a first gas in each of said at least one flow tubes to create a gas-lift flow in said inner lumen; and
(b) a stage two digestion module, comprising:
i) a second digestion vessel having an inlet, an outlet, and a second digestion chamber therebetween, with said second digestion vessel inlet in fluid communication with said first digestion vessel outlet, said second digestion chamber having an upper portion and a lower portion;
ii) at least one flow tube positioned in said second digestion chamber and dividing said chamber into an inner lumen and an outer lumen, said flow tube configured to allow current flow of said organic material between said outer lumen and said inner lumen; and
iii) a second gas source connected to said second digestion chamber and configured to bubble a second gas in each of said at least one flow tubes to create a gas-lift flow in said inner lumen.

2. The device of claim 1, wherein said at least one flow tube of said stage one digestion module or said stage two digestion module is configured to allow current flow of said organic material from said outer lumen to said inner lumen at said first digestion chamber bottom portion, and current flow from said inner lumen to said outer lumen at said first digestion chamber upper portion.

3. The device of claim 2, wherein said current flow is laminar flow.

4. The device of claim 1, wherein said first gas source is connected to said first digestion chamber lower portion beneath each of said at least one flow tubes to create a gas-lift flow in said inner lumen.

5. The device of claim 1, wherein said second gas source is connected to said second digestion chamber lower portion beneath each of said at least one flow tubes to create a gas-lift flow in said inner lumen.

6. The device of claim 1, wherein said stage one digestion module and/or said stage two digestion module further comprises a foam collector configured to accept foam that develops on the surface of said organic material at the top portion of said at least one chamber.

7. The device of claim 6, wherein said foam collector comprises a foam outlet and a gas inlet, and wherein said stage one digestion module and/or said stage two digestion module further comprises a foam separator in fluid communication with said foam outlet, said foam separator comprising a top portion and a bottom portion, said top portion having a gas outlet in gas communication with said gas inlet of said foam collector, and said bottom portion having a liquid outlet, said liquid outlet in fluid communication with said first or second digestion vessel.

8. The device of claim 1, further comprising a water jacket surrounding said first digestion vessel and said second digestion vessel.

9. The device of claim 8, further comprising a heater operatively connected to said water jacket.

10. The device of claim 1, further comprising a heat-exchanger operatively associated with said first gas source and configured for distillation of said first gas.

11. The device of claim 1, further comprising a heat-exchanger operatively associated with said second gas source and configured for distillation of said second gas.

12. The device of claim 1, wherein said stage one digestion module and said stage two digestion module each comprises at least three digestion vessels arranged in series.

13. A method of digesting organic material, comprising digesting said material using the device of claim 1.

14. A method of digesting organic material and creating methane comprising:
providing a multi-stage plug-flow gas-lift digestion device of claim 1;
loading said organic material into said inlet of said stage one digestion module;
mixing the organic material by bubbling said first gas in said first reaction vessel;
overfilling said reaction chamber of said first reaction vessel so that the organic material spills into one or more subsequent chambers, while digesting said organic material to create acetate, and said acetate created thereby flowing into said first module outlet;
passing said acetate through said stage one digestion module outlet and into said stage two digestion module inlet;
mixing the acetate by bubbling said second gas in second reaction vessel; and
overfilling said reaction chamber of said second reaction vessel so that acetate spills into a subsequent chamber or outlet, while digesting said acetate to form said methane;
to thereby digest said organic material and create methane.

15. The method of claim 14, further comprising providing in said device and/or in said organic material a bacterial mixture capable of digesting said organic material and capable of producing methane from acetate.

16. The method of claim 15, further comprising:
providing temperature and pH conditions in said stage one digestion module conducive to digestion of said organic material by said bacterial mixture; and
providing temperature and pH conditions in said stage two digestion module conducive to production of said methane by said bacterial mixture from said acetate.

17. The method of claim 17 wherein, prior to said loading of said organic material into said inlet of said stage one digestion module, said method further comprises a step selected from the group consisting of:
(a) alkalinizing said organic material to thereby saponify ester linkages therein;
(b) acidifying said organic material to thereby hydrolyze peptide linkages;
(c) heating said organic material to facilitate said digesting;
(d) exposing said organic material to electromagnetic energy to break aromatic linkages in said organic material; and
(e) a combination thereof.

18. A method of collecting a biofuel comprising:
providing a multi-stage plug-flow gas-lift digestion device of claim 10;
loading said organic material into said inlet of said stage one digestion module;
mixing the organic material by bubbling said first gas in said first reaction vessel;
overfilling said reaction chamber of said first reaction vessel so that the organic material spills into one or more subsequent chambers, while digesting said organic material to create acetate, and said acetate created thereby flowing into said first module outlet;
passing said acetate through said stage one digestion module outlet and into said stage two digestion module inlet;
mixing the acetate by bubbling said second gas in second reaction vessel; and
overfilling said reaction chamber of said second reaction vessel so that acetate spills into a subsequent chamber or outlet, while digesting said acetate to form methane; and
collecting a distillate from said heat exchanger operatively associated with said first gas source or from said heat exchanger operatively associated with said second gas, said distillate comprising said biofuel,
to thereby collect said biofuel.

19. The method of claim 18, wherein said biofuel is an alcohol.

20. The method of claim 12, wherein the organic material comprises municipal, industrial, agricultural or domestic wastes.

21. The method of claim 12, wherein the organic material comprises swine fecal waste.
